(19) United States
(12) Patent Application Publication
(10) Pub. No.: US 2007/0087392 A1
(43) Pub. Date: Apr. 19, 2007

(54) Method for diagnosing head and neck squamous cell carcinoma

(75) Inventors: Kenneth D. Somers, Richmond, VA (US); Bao-Ling Adam, Norfolk, VA (US); George L. Wright JR., Virginia Beach, VA (US); Jeffrey T. Wadsworth, Suffolk, VA (US)

Correspondence Address: WILMER CUTLER PICKERING HALE AND DORR LLP, 1875 PENNSYLVANIA AVE., NW, WASHINGTON, DC, 20004, US

(73) Assignee: Eastern Virginia Medical School, Norfolk, VA (US)

(21) Appl. No.: 10/569,078

(22) PCT Filed: Aug. 19, 2004
(86) PCT No.: PCT/US04/26872

§ 371 Date: Dec. 18, 2006

Related U.S. Application Data
(60) Provisional application No. 60/495,878, filed on Aug. 19, 2003.
(60) Provisional application No. 60/496,682, filed on Aug. 21, 2003.

(51) Int. Cl. G01N 33/574

(52) U.S. Cl. 435/7.23

(57) ABSTRACT

Disclosed are protein biomarkers and their use in diagnosing head and neck squamous cell carcinoma (HNSCC) or to make a negative diagnosis in patients. Also disclosed are kits for the diagnosis of HNSCC that detect the protein biomarkers of the invention, as well as methods using a plurality of classifiers to make a probable diagnosis of HNSCC. In certain aspects of the invention, the methods include use of a decision tree analysis. Various computer readable media and their use according to the invention are also disclosed.

### STATEMENT AS TO RIGHTS TO INVENTIONS MADE UNDER FEDERALLY SPONSORED RESEARCH AND DEVELOPMENT

The present invention was made with Government support under grant number CA85067 awarded by the National Institutes of Health/National Cancer Institute. The Government may have certain rights in the invention.

### BACKGROUND OF THE INVENTION

Head and neck squamous cell carcinoma (HNSCC) remains a significant disease, comprising over 5% of all cancers in the United States and an even larger proportion of cancers worldwide (Jemal, A. et al., CA Cancer J. Clin 53:5-26 (2003)). Well-established risk factors for HNSCC include tobacco use and excessive alcohol consumption. Despite increased awareness of these risk factors, the incidence of HNSCC in the United States has not changed significantly. Furthermore, little progress has been made towards improving survival rates, notwithstanding the many advances in the treatment of HNSCC over the past 30 years.

Detection of head and neck cancer at early disease stages is paramount to successful clinical therapy. However, screening for HNSCC is not even mentioned in the most recent screening guidelines of the American Cancer Society (Smith, R. CA Cancer J Clin. 53: 27-43 (2003)) due primarily to the lack of sufficient screening tools available to physicians. Aside from a complete physical examination of the head and neck and imaging studies in those patients with suspicious clinical findings or symptoms, there are no accepted forms of screening for these cancers. There is currently no standard and effective screening tool available for HNSCC patients. Due to the location of the HNSCC tumors and the fact that early symptoms of HNSCC often mimic benign processes such as viral upper respiratory infections, most patients are not diagnosed until the late stages of the disease, leading to morbidity and a significantly diminished quality of life. For example, treatment of advanced HNSCC frequently leaves patients disfigured. In addition, the debilitating side effects of radiation and chemotherapy result in compromised speech and swallowing.

The search for biomarkers predictive of HNSCC has focused largely on the detection of genetic abnormalities leading to the development of HNSCC (Gleich, L., et al., Cancer Control 9:369-378 (2002); Patel, V., et al., Crit. Rev. Oral Biol. Med. 12:55-63 (2001)). However, despite the identification and characterization of multiple molecular aberrations in HNSCC, available technology limits their routine clinical use and none has been determined to enhance early detection of HNSCC.

A number of studies have also described limited success in identifying HNSCC-associated protein and DNA/RNA biomarkers that may aid in the early diagnosis and prognosis of HNSCC. For instance, RT-PCR was used to detect metastasis-associated cytokeratin 19 positive tumor cells in sera from a small number of patients with nasopharyngeal carcinoma. However, in this study several longitudinal blood samples were required to reach a sensitivity of 83.3% (5 of 6 patients) (Lin, J. C., et al., Head Neck 24:591-596 (2002)). In addition, an ELISA analysis of serum concentrations of multiple biologic markers including basic fibroblast growth factor (bFGF), vascular endothelial growth factor, and matrix metalloproteinase-2 in 26 HNSCC patients following primary chemoradiation therapy, showed that only increased bFGF concentrations correlated with earlier locoregional control (Dietz, A., et al., Head Neck 22:666-673 (2000)). Other studies have investigated several conventional serologic markers in 26 HNSCC patients and found none to be of statistical significance (Walther, E. K., et. al., Head Neck 15:230-235 (1993)). Further studies have found that antibodies to p53 tumor suppressor protein were detected in the sera of 25% of 271 patients with oral SCC (Gottschlich, S., et al., Anticancer Res. 19:2703-2705 (1999)) and at a low percentage in saliva from HNSCC patients (Tavassoli, M., et al., Int J. Cancer 78:390-391 (1998); Warnakulasuriya, S., et al., J. Pathology 192:52-57 (2000)). Nucleic acid-based microsatellite analysis and tumor-specific aberrant promoter methylation have also been used as markers to detect tumor-specific alterations in serum and saliva of patients with HNSCC (Nawroz, H., et al., Nature Med. 2:1035-1037 (1996); Spafford, M. F., Clin. Cancer Res. 7:607-612 (2001); El-Naggar, A. K., et al., J. Molec. Diagnostics 3:164-170 (2001); Sanchez-Cespedes, M., et al., Cancer Res. 60:892-895 (2000); Rosas, S. L. B., et al., Cancer Res. 61:939-942 (2001)). However, these approaches are often subjective, can be technically challenging, and require a panel of microsatellite markers or selected genes. In general, nucleic acid-based methods for detection of cancer have been assessed with a limited number of samples and will require further trials to confirm these early results.

Recently, attention has focused on deciphering the HNSCC proteome in search of diagnostic biomarkers. Assays to detect certain differentially expressed proteins from HNSCC patients are currently being evaluated for their diagnostic and prognostic utility. For instance, assays to detect the epidermal growth factor receptor (EGFr) ectodomain protein (U.S. Pat. No. 5,344,760 to Harvey, et al.), a p16 polypeptide (U.S. Pat. No. 5,856,094 to Sidransky, et al.), metallopanstimulin (“MPS”) related proteins (U.S. Pat. No. 5,955,287 to Fernandez-Pol), and polypeptides comprising at least a portion of a head and neck tumor protein (U.S. Patent Application Publication No. US 2002/0168647 A1 to Wang, et al.), are currently being examined for their effectiveness in diagnosing HNSCC.

In addition to immunoassays, proteomic research has traditionally involved two-dimensional gel electrophoresis (2D-PAGE) to detect protein expression differences in tissue and body fluid specimens between healthy (control) groups and disease groups (Srinivas, P. R., et al., Clin Chem. 47:1901-1911 (2001); Adam, B. L., et al., Proteomics 1:1264-1270 (2001)). Although two-dimensional polyacrylamide gel electrophoresis (2D-EP) has been the classical approach in exploring the proteome for separation and detection of differences in protein expression, it has its limitations in that it is cumbersome, labor intensive, suffers reproducibility problems, and is not easily applied in the clinical setting.

Overall, despite the identification and extensive study of several potential tumor markers, none has been found to have clinical utility as a diagnostic marker or screening tool for HNSCC. It seems probable that given the complexity of the genetic and molecular alterations that occur in HNSCC cells, the expression pattern of these complex changes may hold more vital information in screening, diagnosis and prognosis than the individual molecular changes themselves.

One of the recent technological advances in proteomics is the Proteinchip® surface-enhanced laser desorption/ionization time of flight mass spectrometry (SELDI-TOF-MS) (Kuwata, H., et al., Biochem. Biophys. Res. Commun. 245:764-773 (1998); Merchant, M. et al., Electrophoresis 21:1164-1177 (2000)). This system uses surface-enhanced laser desorption/ionization time-of-flight (SELDI-TOP) mass spectrometry to detect proteins bound to a protein chip array. The SELDI system is an extremely sensitive and rapid method that analyzes complex mixtures of proteins and peptides. Applications of this technology show great potential for the early detection of prostate, breast, ovarian, and bladder cancers (Li, J., et al., Clin. Chem. 48:1296-1304 (2002); Adam, B., et al., Cancer Res. 62:3609-3614 (2002); Cazares, L. H., et al., Clin. Cancer Res. 8:2541-2552 (2002); Petricoin, E. F., et al., Lancet 359:572-577 (2002); Petricoin, E. F. et al., J. Natl. Cancer Inst. 94:1576-1578 (2002); Vlahou, A., et al., Amer. J. Pathology 158:1491-1502 (2001)). Proteinchip® technology has been used to detect a 8670 Dalton protein in tumor extracts from five of six HNSCC cases, not found in matched normal tissue lysates (von Eggeling, F, et al., BioTechniques 29:1066-1070 (2000)). The SELDI technology has also been used to detect differential expression of proteins in two HNSCC cell lines (one metastatic and one not). In a study of two matched HNSCC cell lines derived from either the primary tumor or lymph node metastasis, the SELDI Proteinchip® H4 was used to identify the up-regulation of two membrane-associated proteins (annexin I and annexin II) and a glycolytic protein (enolase-α) in the metastatic cell line. It also detected the down-regulation of calumenin precursor in the metastatic cell line (Wu, W., et al., Clin. Exp. Metastasis 19:319-326 (2002)). To date, however, SELDI Proteinchip® technology has not been reported as a tool of interrogation for serum from HNSCC patients compared to normal controls in order to develop HNSCC protein fingerprints.

Continued efforts to identify protein profiles or patterns that differentiate cancer from non-cancer could lead to earlier detection and development of diagnostic tests for HNSCC. There is a need, then, for methods and compositions for the diagnosis of HNSCC that can be performed relatively fast and inexpensively, yet are clinically useful. The present invention addresses this and other needs.

### SUMMARY OF THE INVENTION

The present invention provides, for the first time, novel protein markers that are differentially present in the samples of patients with head and neck squamous cell carcinoma (HNSCC) and in the samples of control subjects. The present invention also provides sensitive and quick methods and kits that can be used as an aid for the diagnosis of HNSCC by detecting these novel markers. The measurement of these markers, alone or in combination, in patient samples, provides information that can be correlated with a probable diagnosis of HNSCC or a negative diagnosis (e.g., normal or disease-free). All the markers are characterized by molecular weight. The markers can be resolved from other proteins in a sample by, e.g., chromatographic separation coupled with mass spectrometry, or by traditional immunoassays. In preferred embodiments, the method of resolution involves Surface-Enhanced Laser Desorption/Ionization (“SELDI”) mass spectrometry, in which the surface of the mass spectrometry probe comprises absorbents that bind to the marker.

In one form of the invention, a method for aiding in, or otherwise making, a diagnosis includes detecting at least one protein biomarker in a test sample from a subject. The protein biomarkers have a molecular weight selected from the group consisting of about 2778±5.6, 2951±5.9, 3772±7.5, 3888±7.8, 4181±8.4, 4464±8.9, 5064±10.1, 5078±10.2, 5242±10.5, 5335±10.7, 5363±10.7, 5544±11.1, 5905±11.8, 5920±11.8, 6110±12.2, 7764±15.5, 7805±15.6, 7830±15.7, 7920±15.8, 7971±15.9, 8928±17.9, 9094±18.1, 9134±18.3, 9181±18.4, 9287±18.6, 9416±18.8, 10264±20.5, 10843±21.7, 11722±23.4, 11922±23.8, 13350±26.7, 13881±27.8, 14687±29.4, and 15139±30.3 Daltons. The method further includes correlating the detection with a probable diagnosis of HNSCC or a negative diagnosis.

In one embodiment, the correlation takes into account the amount of the marker or markers in the sample and/or the frequency of detection of the same marker or markers in a control.

In another embodiment, gas phase ion spectrometry is used for detecting the marker or markers. For example, laser desorption/ionization mass spectrometry can be used.

In another embodiment, laser desorption/ionization mass spectrometry used to detect markers comprises: (a) providing a substrate comprising an adsorbent attached thereto; (b) contacting the sample with the adsorbent; and (c) desorbing and ionizing the marker or markers with the mass spectrometer. Any suitable adsorbent can be used to bind one or more markers. For example, the adsorbent on the substrate can be a cationic adsorbent, an antibody adsorbent, etc.

In another embodiment, an immunoassay can be used for detecting the marker or markers.

In certain forms of the invention, the markers in the test sample from a subject may be detected in the following groups and may have the following molecular weights: about 5064, 13881, and 15139 Daltons.

In accordance with the present invention, at least one of the biomarkers described herein may be detected. It is to be understood, and is described herein, that one or more of the biomarkers may be detected and subsequently analyzed, including all of the biomarkers. Further, it is to be understood that the failure to detect one or more of the biomarkers of the invention, or the detection thereof at levels or quantities that may correlate with the absence of clinical or pre-clinical HNSCC, may be useful and desirable as a means of diagnosing the absence of clinical or pre-clinical HNSCC, and that the same forms a contemplated aspect of the present invention.

In yet another aspect of the invention, kits that may be utilized to detect the biomarkers described herein and may otherwise be used to diagnose, or otherwise aid in the diagnosis of HNSCC, are provided. In one form of the invention, a kit may include a substrate comprising an adsorbent attached thereto, wherein the adsorbent is capable of retaining at least one protein biomarker selected from the group consisting of about 5064±10.1, 13881±27.8, and 15139±30.3 Daltons; and instructions to detect the protein biomarker by contacting a test sample with the adsorbent and detecting the biomarker retained by the adsorbent.

In yet another embodiment of the invention, the kit may include a substrate comprising an adsorbent attached thereto, wherein the adsorbent is capable of retaining at least one protein biomarker selected from the group consisting of about 2778±5.6, 2951±5.9, 3772±7.5, 3888±7.8, 4181±8.4, 4464±8.9, 5064±10.1, 5078±10.2, 5242±10.5, 5335±10.7, 5363±10.7, 5544±11.1, 5905±11.8, 5920±11.8, 6110±12.2, 7764±15.5, 7805±15.6, 7830±15.7, 7920±15.8, 7971±15.9, 8928±17.9, 9094±18.1, 9134±18.3, 9181±18.4, 9287±18.6, 9416±18.8 10264±20.5, 10843±21.7, 11722±23.4, 11922±23.8, 13350±26.7, 13881±27.8, 14687±29.4, and 15139±30.3 Daltons; and instructions to detect the protein biomarker by contacting a test sample with the adsorbent and detecting the biomarker retained by the adsorbent.

In yet another aspect of the invention, methods of using a plurality of classifiers to make a probable diagnosis of HNSCC or a negative diagnosis are provided. In one form of the invention, a method includes a) obtaining mass spectra from a plurality of samples from normal subjects and subjects diagnosed with HNSCC; b) applying a decision tree analysis to at least a portion of the mass spectra to obtain a plurality of weighted base classifiers comprising a peak intensity value and an associated threshold value; and c) making a probable diagnosis of HNSCC or a negative diagnosis based on a linear combination of the plurality of weighted base classifiers. In certain forms of the invention, the method may include using the peak intensity value and the associated threshold value in linear combination to make a probable diagnosis of HNSCC or to make a negative diagnosis.

It is a further object of the invention to provide computer program media storing computer instructions therein for instructing a computer to perform a computer-implemented process for developing and/or using a plurality of classifiers to make a probable diagnosis of HNSCC or a negative diagnosis using at least one protein biomarker selected from the group consisting of about 2778±5.6, 2951±5.9, 3772±7.5, 3888±7.8, 4181±8.4, 4464±8.9, 5064±10.1, 5078±10.2, 5242±10.5, 5335±10.7, 5363±10.7, 5544±11.1, 5905±11.8, 5920±11.8, 6110±12.2, 7764±15.5, 7805±15.6, 7830±15.7, 7920±15.8, 7971±15.9, 8928±17.9, 9094±18.1, 9134±18.3, 9181±18.4, 9287±18.6, 9416±18.8, 10264±20.5, 10843±21.7, 11722±23.4, 11922±23.8, 13350±26.7, 13881±27.8, 14687±29.4, and 15139±30.3 Daltons. Preferably, the protein biomarkers are selected from the about 5064±10.1, 13881±27.8, and 15139±30.3 Daltons protein biomarkers.

### BRIEF DESCRIPTION OF THE FIGURES

FIG. 1 depicts a schematic of the decision tree classification system utilized in Example 1. “HC” represents healthy control patients while “HNSCC” represents head and neck squamous cell patients. The squares are the primary nodes and the circles indicate terminal nodes. The mass value in the root nodes are followed by ≦ the intensity value.

FIG. 2 shows a representative SELDI gel view (A) and spectra (B) from sera of six HNSCC patients compared with sera from six normal controls ranging from 4,000 to 6,000 m/z. The “box” identifies a peak with an average mass of 5064 Da that is underexpressed in HNSCC compared to normal serum.

FIG. 3 shows the expression level of the 5064 Da protein in the sera of HNSCC patients compared with sera from normal controls. “−” indicates the mean normalized intensity and “◯” indicates values of individual patients.

FIG. 4 depicts representative SELDI spectra of two different serum samples assayed three months apart using the IMAC Proteinchip®.

FIG. 5 illustrates one example of a central processing unit for implementing a computer process in accordance with a computer implemented embodiment of the present invention.

FIG. 6 illustrates one example of a block diagram of internal hardware of the central processing unit of FIG. 5.

FIG. 7 is an illustrative computer-readable medium upon which computer instructions can be embodied.

### DESCRIPTION OF THE PREFERRED EMBODIMENTS

For the purposes of promoting an understanding of the principles of the invention, reference will now be made to preferred embodiments and specific language will be used to describe the same. It will nevertheless be understood that no limitation of the scope of the invention is thereby intended, such alteration and further modifications of the invention, and such further applications of the principles of the invention as illustrated herein, being contemplated as would normally occur to one skilled in the art to which the invention relates.

The present invention relates to methods for aiding in a diagnosis of, and methods for diagnosing HNSCC. Surface enhanced laser desorption/ionization mass spectroscopy has been combined with various algorithms to deduce protein biomarkers that may be utilized in various decision trees to aid in the diagnosis of, and/or to diagnose HNSCC or to make a negative diagnosis. Accordingly, such protein biomarkers are also provided herein.

The methods of the present invention effectively differentiate between individuals with HNSCC and normal individuals. As defined herein, normal individuals are individuals with a negative diagnosis with respect to HNSCC. That is, normal individuals do not have HNSCC. The method includes detecting a protein biomarker in a test sample from a subject. For example, the protein biomarkers having a molecular weight of about 2778±5.6, 2951±5.9, 3772±7.5, 3888±7.8, 4181±8.4, 4464±8.9, 5064±10.1, 5078±10.2, 5242±10.5, 5335±10.7, 5363±10.7, 5544±11.1, 5905±11.8, 5920±11.8, 6110±12.2, 7764±15.5, 7805±15.6, 7830±15.7, 7920±15.8, 7971±15.9, 8928±17.9, 9094±18.1, 9134±18.3, 9181±18.4, 9287±18.6, 9416±18.8, 10264±20.5, 10843±21.7, 11722±23.4, 11922±23.8, 13350±26.7, 13881±27.8, 14687±29.4, and 15139±30.3 Daltons have been identified that aid in the probable diagnosis of HNSCC or aid in a negative diagnosis. In accordance with the present invention, at least one of the protein biomarkers is detected. Preferably, two or more, three or more, four or more, five or more, ten or more, fifteen or more, twenty or more, thirty or more, or all thirty-four protein biomarkers are detected and the presence or absence of such biomarkers is correlated to a diagnosis of HNSCC. As used herein, the term “detecting” includes determining the presence, the absence, the quantity, or a combination thereof, of the protein biomarkers. The quantity of the biomarkers may be represented by the peak intensity as identified by mass spectrometry, for example, or concentration of the biomarkers.

In certain forms of the invention, selected groups of protein biomarkers find utility in diagnosing HNSCC. For example, the following groups of markers find utility in making, or otherwise aiding in making, a specific diagnosis: (1) the about 5064±10.1 Dalton biomarker, (2) the about 5064±10.1 and 13881±27.8 Dalton biomarkers; (3) the about 5064±10.1, 13881±27.8, and 15139±30.3 Dalton biomarkers; (4) the about 2778±5.6, 3772±7.5, 4464±8.9, 5064±10.1 Dalton biomarkers, and (5) the about 5064±10.1, 5242±10.5, 13881±27.8, and 15139±30.3 Dalton biomarkers. Preferably, the about 5064±10.1 Dalton biomarker or the combination of the about 5064±10.1, 13881±27.8, and 15139±30.3 Dalton biomarkers is used.

The differential expression, such as the over- or under-expression, of selected biomarkers relative to normal individuals may be correlated to HNSCC. By differentially expressed, it is meant herein that the protein biomarkers may be found at a greater or smaller level in one disease state compared to another, or that it may be found at a higher frequency in one or more disease state. For example, the underexpression of the about 5064±10.1 Dalton biomarker by at least two-fold, preferably three-fold, relative to the normal patient may be correlated with the probable diagnosis of HNSCC. Moreover, the about 2778±5.6, 2951±5.9, 3772±7.5, 3888±7.8, 4181±8.4, 4464±8.9, 5064±10.1, 5078±10.2, 5242±10.5, 5335±10.7, 5363±10.7, 5544±11.1, 5905±11.8, 5920±11.8, 6110±12.2, 7764±15.5, 7805±15.6, 7830±15.7, 7920±15.8, 7971±15.9, 8928±17.9, 9094±18.1, 9134±18.3, 9181±18.4, 9287±18.6, 9416±18.8, 10264±20.5, 10843±21.7, 11722±23.4, 11922±23.8, 13350±26.7, 14687±29.4 Dalton biomarkers have been found to be differentially expressed in HNSCC patients relative to normal patients. In particular, the about 2778, 3772, 4464, 8928, 9094, 9134, 11722, 11922, 13350, and 14687 Dalton biomarkers have been found to be overexpressed in HNSCC patients and the about 3888, 5064, 5078, 5335, 5905, 6110, 7764, 7805, 7920, and 7971 Dalton biomarkers have been found to be under-expressed in HNSCC patients.

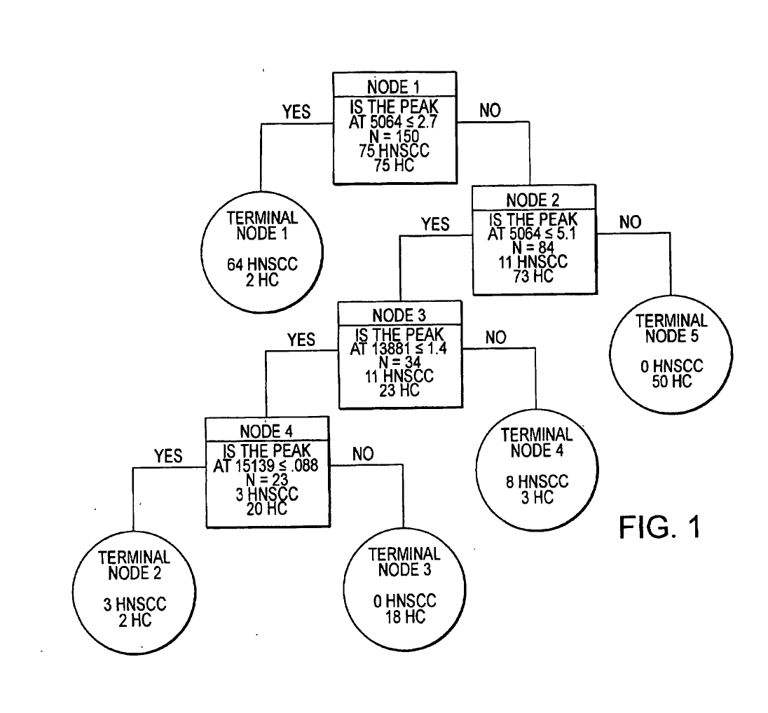

Moreover, combinations of groupings of biomarkers in classification trees have been found to be useful to identify HNSCC-positive and HNSCC-negative patients. For example, FIG. 1 depicts a suitable classification tree that may be used to distinguish HNSCC and normal patients. In one group, the presence of the about 5064±10.1 Dalton biomarker at a threshold value of less than or equal to 2.7 may be correlated to a diagnosis of HNSCC. In another group, the presence of the about 5064±10.1 Dalton biomarker at a peak intensity threshold value of greater than 2.7, but less than or equal to 5.1, and the presence of the about 13881±27.8 Dalton biomarker at a peak intensity of greater than 1.4 may be correlated to a probable diagnosis of HNSCC. In another group, the presence of the about 5064±10.1 Dalton biomarker at a peak intensity threshold value of greater than 2.7, but less than or equal to 5.1, and the presence of the about 13881±27.8 Dalton biomarker at a peak intensity of less than or equal to about 1.4, and the absence of the about 15139±30.3 Dalton biomarker at a threshold of less than or equal to about 0.088 may be correlated to a normal diagnosis. Finally, the presence of the about 5064±10.1 Dalton biomarker at a peak intensity threshold value of greater than about 2.7, but less than or equal to about 5.1, and the presence of the about 13881±27.8 Dalton biomarker at a peak intensity of less than or equal to about 1.4, and the presence of the about 15139±30.3 Dalton biomarker at a threshold of less than or equal to about 0.088 may be correlated to either a HNSCC or normal diagnosis. Preferably, the combination of these groupings makes up a single classification tree for a diagnosis of HNSCC. However, the present invention contemplates the use of these individual groupings alone or in combination with other groupings to aid in the diagnosis or identification of HNSCC-positive and HNSCC-negative patients. Thus, one or more of such groupings, preferably two or more, or more preferably, all of these groupings aid in the diagnosis.

Data analysis can include the steps of determining signal strength (e.g., height of peaks, area of peaks) of a biomarker detected and removing “outerliers” (data deviating from a predetermined statistical distribution). For example, the observed peaks can be normalized, a process whereby the height of each peak relative to some reference is calculated. For example, a reference can be background noise generated by instrument and chemicals (e.g., energy absorbing molecule) which is set as zero in the scale. The signal strength can then be detected for each biomarker or other substances can be displayed in the form of relative intensities in the scale desired (e.g., 100). Alternatively, a standard may be included with the sample so that a peak from the standard can be used as a reference to calculate relative intensities of the signals observed for each biomarker or other markers detected.

The threshold values in FIG. 1 represent the normalized peak intensity of the biomarkers. As more fully described in Example 1, these threshold values may represent the normalized peak intensity of a particular biomarker which is related to the concentration of the biomarker. The normalization process may involve using the total ion current as a normalization factor. The normalization process could alternatively involve reporting the peak intensity relative to the peak intensity of an internal or external control. For example, a known protein may be added to the system. Additionally, a known product produced by the test subject, such as albumin, may act as an internal standard or control. It is understood that the threshold values identified in FIG. 1 are relative to the control used in Example 1. However, as one having ordinary skill in the art would appreciate, this threshold may be different based on the internal or external control.

The method includes detecting at least one protein biomarker. However, any number of biomarkers may be detected. It is preferred that at least two protein biomarkers are detected in the analysis. However, it is realized that three, four, or more, including all, of the biomarkers described herein may be utilized in the diagnosis. Thus, not only can one or more markers be detected, one to 34, preferably two to 34, two to 20, and two to 10 biomarkers, two to 5 biomarkers, or some other combination, may be detected and analyzed as described herein. In addition, other protein biomarkers not herein described may be combined with any of the presently disclosed protein biomarkers to aid in the diagnosis of HNSCC. For instance, the methods of the present invention may be used to detect known biomarkers (i.e., MPS-1 with a molecular weight of about 10,068 Da). These known biomarkers may also be combined with any of the presently disclosed protein biomarkers to aid in the diagnosis of HNSCC. Moreover, any combination of the above protein biomarkers may be detected in accordance with the present invention.

The detection of the protein biomarkers described herein in a test sample may be performed in a variety of ways. In one form of the invention, a method for detecting the biomarker includes detecting the biomarker by gas phase ion spectrometry utilizing a gas phase ion spectrometer. The method may include contacting a test sample having a biomarker, such as the protein biomarkers described herein, with a substrate comprising an adsorbent thereon under conditions to allow binding between the biomarker and adsorbent and detecting the biomarker bound to the adsorbent by gas phase ion spectrometry.

A wide variety of adsorbents may be used. The adsorbents may include a hydrophobic group, a hydrophilic group, a cationic group, an anionic group, a metal ion chelating group, or antibodies that specifically bind to an antigenic biomarker, or some combination thereof (such as a “mixed mode” adsorbent). Exemplary adsorbents that include a hydrophobic group include matrices having aliphatic hydrocarbons, such as C₁-C₁₈ aliphatic hydrocarbons and matrices having aromatic hydrocarbon functional groups, including phenyl groups. Exemplary adsorbents that include a hydrophilic group include silicon oxide, or hydrophilic polymers such as polyalkylene glycol, polyethylene glycol, dextran, agarose or cellulose. Exemplary adsorbents that include a cationic group include matrices of secondary, tertiary or quaternary amines. Exemplary adsorbents that have an anionic group include matrices of sulfate anions and matrices of carboxylate anions or phosphate anions. Exemplary adsorbents that have metal chelating groups include organic molecules that have one or more electron donor groups which may form coordinate covalent bonds with metal ions, such as copper, nickel, cobalt, zinc, iron, aluminum and calcium. Exemplary adsorbents that include an antibody include antibodies that are specific for any of the biomarkers provided herein and may be readily made by methods known to the skilled artisan.

Alternatively, the substrate can be in the form of a probe, which may be removably insertable into a gas phase ion spectrometer. For example, a substrate may be in the form of a strip with adsorbents on its surface. In yet other forms of the invention, the substrate can be positioned onto a second substrate to form a probe which may be removably insertable into a gas phase ion spectrometer. For example, the substrate can be in the form of a solid phase, such as a polymeric or glass bead with a functional group for binding the marker, which can be positioned on a second substrate to form a probe. The second substrate may be in the form of a strip, or a plate having a series of wells at predetermined locations. In this manner, the biomarker can be adsorbed to the first substrate and transferred to the second substrate which can then be submitted for analysis by gas phase ion spectrometry.

The probe can be in the form of a wide variety of desired shapes, including circular, elliptical, square, rectangular, or other polygonal or other desired shape, as long as it is removably insertable into a gas phase ion spectrometer. The probe is also preferably adapted or otherwise configured for use with inlet systems and detectors of a gas phase ion spectrometer. For example, the probe can be adapted for mounting in a horizontally and/or vertically translatable carriage that horizontally and/or vertically moves the probe to a successive position without requiring, for example, manual repositioning of the probe.

The substrate that forms the probe can be made from a wide variety of materials that can support various adsorbents. Exemplary materials include insulating materials, such as glass and ceramic; semi-insulating materials, such as silicon wafers; electrically-conducting materials (including metals such as nickel, brass, steel, aluminum, gold or electrically-conductive polymers); organic polymers; biopolymers, or combinations thereof.

In other embodiments of the invention, depending on the nature of the substrate, the substrate surface may form the adsorbent. In other cases, the substrate surface may be modified to incorporate thereon a desired adsorbent. The surface of the substrate forming the probe can be treated or otherwise conditioned to bind adsorbents that may bind markers if the substrate cannot bind biomarkers by itself. Alternatively, the surface of the substrate can also be treated or otherwise conditioned to increase its natural ability to bind desired biomarkers. Other probes suitable for use in the invention may be found, for example, in PCT international publication numbers WO 01/25791 (Tai-Tung et al.) and WO 01/71360 (Wright et al.).

The adsorbents may be placed on the probe substrate in a wide variety of patterns, including a continuous or discontinuous pattern. A single type of adsorbent, or more than one type of adsorbent, may be placed on the substrate surface. The patterns may be in the form of lines, curves, such as circles, or any such other shape or pattern as desired.

The method of production of the probes will depend on the selection of substrate materials and/or adsorbents as known in the art. For example, if the substrate is a metal, the surface may be prepared depending on the adsorbent to be applied thereon. For example, the substrate surface may be coated with a material, such as silicon oxide, titanium oxide or gold, that allows derivatization of the metal surface to form the adsorbent. The substrate surface may then be derivatized with a bifunctional linker, one of which binds, such as covalently binds, with a functional group on the surface and the opposing end of the linker may be further derivatized with groups that function as an adsorbent. As a further example, a substrate that includes a porous silicon surface generated from crystalline silicon can be chemically modified to include adsorbents for binding markers. Additionally, adsorbents with a hydrogel backbone can be formed directly on the substrate surface by in situ polymerization of a monomer solution which includes, for example, substituted acrylamide or acrylate monomers, or derivatives thereof that include a functional group of choice as adsorbent.

In preferred forms of the invention, the probe may be a chip, such as those available from Ciphergen Biosystems, Inc. (Palo Alto, Calif.). The chip may be a hydrophilic, hydrophobic, anion-exchange, cation-exchange, immobilized metal affinity or preactivated protein chip array. The hydrophobic chip may be a Proteinchip® H4, which includes a long-chain aliphatic surface that binds proteins by reverse phase interaction. The hydrophilic chip may be Proteinchips® NP1 and NP2 which include a silicon dioxide substrate surface. The cation exchange Proteinchip® array may be Proteinchip® WCX2, a weak cation exchange array with a carboxylate surface to bind cationic proteins. Alternatively, the chip may be an anion exchange protein chip array, such as SAX1 (strong anion exchange) Proteinchip® which is made from silicon-dioxide-coated aluminum substrates, or Proteinchip® SAX2 with a higher capacity quaternary ammonium surface to bind anionic proteins. A further useful chip may be the immobilized metal affinity capture chip (IMAC3) having nitrilotriacetic acid on the surface. Further alternatively, Proteinchip® PS1 is available which includes a carbonyldiimidazole surface which covalently reacts with amino groups or may be Proteinchip® PS2 which includes an epoxy surface which covalently reacts with amine and thiol groups.

In accordance with the present invention, the probe contacts a test sample. The test sample may be obtained from a wide variety of sources. The sample is typically obtained from biological fluid from a subject or patient who is being tested for HNSCC or from a normal individual who may be thought to be of risk for the disease. A preferred biological fluid is blood or blood sera. Other biological fluids in which the biomarkers may be found include, for example, seminal fluid, seminal plasma, lymph fluid, lung/bronchial washes, mucus, nipple secretions, sputum, tears, saliva, urine, or other similar fluid. If necessary, the sample can be solubilized in or mixed with an eluant prior to being contacted with the probe. The probe may contact the test sample solution by a wide variety of techniques, including bathing, soaking, dipping, spraying, washing, pipetting or other desirable methods. The method is performed so that the adsorbent of the probe preferably contacts the test sample solution. Although the concentration of the biomarker or biomarkers in the sample may vary, it is generally desirable to contact a volume of test sample that includes about 1 attomole to about 100 picomoles of marker in about 1 μl to about 500 μl solution for binding to the adsorbent.

The sample and probe contact each other for a period of time sufficient to allow the biomarker to bind to the adsorbent. Although this time may vary depending on the nature of the sample, the nature of the biomarker, the nature of the adsorbent and the nature of the solution the biomarker is dissolved in, the sample and adsorbent are typically contacted for a period of about 30 seconds to about 12 hours, preferably about 30 seconds to about 15 minutes.

The temperature at which the probe contacts the sample will depend on the nature of the sample, the nature of the biomarker, the nature of the adsorbent and the nature of the solution the biomarker is dissolved in. Generally, the sample may be contacted with the probe under ambient temperature and pressure conditions. However, the temperature and pressure may vary as desired. In presently preferred embodiments of the invention, for example, the temperature may vary from about 4° C. to about 37° C.

After the sample has contacted the probe for a period of time sufficient for the marker to bind to the adsorbent or substrate surface should no adsorbent be used, unbound material may be washed from the substrate or adsorbent surface so that only bound materials remain on the respective surface. The washing can be accomplished by, for example, bathing, soaking, dipping, rinsing, spraying or otherwise washing the respective surface with an eluant or other washing solution. A microfluidics process is preferably used when a washing solution such as an eluant is introduced to small spots of adsorbents on the probe. The temperature of the washing solution may vary, but is typically about 0° C. to about 100° C., and preferably about 4° C. and about 37° C.

A wide variety of washing solutions may be utilized to wash the probe substrate surface. The washing solutions may be organic solutions or aqueous solutions. Exemplary aqueous solutions may be buffered solutions, including HEPES buffer, a Tris buffer, phosphate buffered saline or other similar buffers known to the art. The selection of a particular washing solution will depend on the nature of the biomarkers and the nature of the adsorbent utilized. For example, if the probe includes a hydrophobic group and a sulfonate group as adsorbents, such as the SCXI Proteinchip® array, then an aqueous solution, such as a HEPES buffer, may be used. As a further example, if a probe includes a metal binding group as an adsorbent, such as with the Ni(II) Proteinchip® array, than an aqueous solution, such as a phosphate buffered saline may be preferred. As yet a further example, if a probe includes a hydrophobic group as an adsorbent, such as with the HF Proteinchip® array, water may be a preferred washing solution.

An energy absorbing molecule, such as one in solution, may be applied to the markers or other substances bound on the substrate surface of the probe. As used herein, an “energy absorbing molecule” refers to a molecule that absorbs energy from an energy source in a gas phase ion spectrometer, which may assist the desorption of markers or other substances from the surface of the probe. Exemplary energy absorbing molecules include cinnamic acid derivatives, sinapinic acid, dihydroxybenzoic acid and other similar molecules known to the art. The energy absorbing molecule may be applied by a wide variety of techniques previously discussed herein for contacting the sample and probe substrate, including, for example, spraying, pipetting or dipping, preferably after the unbound materials are washed off the probe substrate surface.

After the biomarker is appropriately bound to the probe, the biomarker may be detected, quantified and/or its characteristics may be otherwise determined using an appropriate detection instrument, preferably a gas phase ion spectrometer. As known in the art, gas phase ion spectrometers include, for example, mass spectrometers, ion mobility spectrometers, and total ion current measuring devices.

In a preferred embodiment, a mass spectrometer is utilized to detect the biomarkers bound to the substrate surface of the probe. The probe, with the bound marker on its surface, may be introduced into an inlet system of the mass spectrometer. The marker may then be ionized by an ionization source, such as a laser, fast atom bombardment, plasma or other suitable ionization sources known to the art. The generated ions are typically collected by an ion optic assembly and a mass analyzer then disperses and analyzes the passing ions. The ions exiting the mass analyzer are detected by a detector. The detector translates information of the detected ions into mass-to-charge ratios. Detection and/or quantitation of the marker will typically involve detection of signal intensity.

In further preferred forms of the invention, the mass spectrometer is a laser desorption time-of-flight mass spectrometer, and further preferably surface enhanced laser desorption time-of-flight mass spectrometry (SELDI) is utilized. SELDI is an improved method of gas phase ion spectrometry for biomolecules. In SELDI, the surface on which the analyte is applied plays an active role in the analyte capture and/or desorption.

As known in the art, in laser desorption mass spectrometry, a probe with a bound marker is introduced into an inlet system. The marker is desorbed and ionized into the gas phase by a laser ionization source. The ions generated are collected by an ion optic assembly. Ions are accelerated in a time-of-flight mass analyzer through a short high voltage field and allowed to drift into a high vacuum chamber. The accelerated ions strike a sensitive detector surface at a far end of the high vacuum chamber at a different time. As the time-of-flight is a function of the mass of the ions, the elapsed time between ionization and impact can be used to identify the presence or absence of molecules of specific mass. Quantitation of the biomarkers, either in relative or absolute amounts, may be accomplished by comparison of the intensity of the displayed signal of the biomarker to a control amount of a biomarker or other standard as known in the art. The components of the laser desorption time-of-flight mass spectrometer may be combined with other components described herein and/or known to the skilled artisan that employ various means of desorption, acceleration, detection, or measurement of time.

In further embodiments, detection and/or quantitation of the biomarkers may be accomplished by matrix-assisted laser desorption ionization (MALDI). MALDI also provides for vaporization and ionization of biological samples from a solid-state phase directly into the gas phase. As known in the art, the sample, including the desired analyte, is dissolved or otherwise suspended in, a matrix that co-crystallizes with the analyte, preferably to prevent the degradation of the analyte during the process.

An ion mobility spectrometer can be used to detect and characterize the biomarkers described herein. The principle of ion mobility spectrometry is based on different mobility of ions. Specifically, ions of a sample produced by ionization move at different rates, due to their difference in, for example, mass, charge, or shape, through a tube under the influence of an electric field. The ions (typically in the form of a current) are registered at the detector which can then be used to identify a marker or other substances in the sample. One advantage of ion mobility spectrometry is that it can operate at atmospheric pressure.

A total ion current measuring device can be used to detect and characterize the biomarkers described herein. This device can be used, for example, when the probe has a surface chemistry that allows only a single type of marker to be bound. When a single type of marker is bound on the probe, the total current generated from the ionized biomarker reflects the nature of the marker. The total ion current produced by the biomarker can then be compared to stored total ion current of known compounds. Characteristics of the biomarker can then be determined.

Data generated by desorption and detection of the biomarkers can be analyzed with the use of a programmable digital computer. The computer program generally contains a readable medium that stores codes. Certain code can be devoted to memory that includes the location of each feature on a probe, the identity of the adsorbent at that feature and the elution conditions used to wash the adsorbent. Using this information, the program can then identify the set of features on the probe defining certain selectivity characteristics, such as types of adsorbent and eluants used. The computer also contains code that receives data on the strength of the signal at various molecular masses received from a particular addressable location on the probe as input. This data can indicate the number of biomarkers detected, optionally including the strength of the signal and the determined molecular mass for each biomarker detected. As described above, the data may be normalized according to known methods, such as by determining the signal strength (e.g., height of peaks or area of peaks) of a biomarker detected and removing any “outerliers.”

The computer can transform the resulting data into various formats for displaying. In one format, referred to as “spectrum view or retentate map,” a standard spectral view can be displayed, wherein the view depicts the quantity of biomarker reaching the detector at each particular molecular weight. In another format, referred to as “peak map,” only the peak height and mass information are retained from the spectrum view, yielding a cleaner image and enabling markers with nearly identical molecular weights to be more easily seen. In yet another format, referred to as “gel view,” each mass from the peak view can be convened into a grayscale image based on the height of each peak, resulting in an appearance similar to bands on electrophoretic gels. In a further format, referred to as “3-D overlays,” several spectra can be overlayed to study subtle changes in relative peak heights. In yet a further format, referred to as “difference map view,” two or more spectra can be compared, conveniently highlighting unique biomarkers and biomarkers which are up- or down-regulated between samples. Biomarker profiles (spectra) from any two samples may be compared visually.

Using any of the above display formats, it can be readily determined from the signal display whether a biomarker having a particular molecular weight is detected from a sample. Moreover, from the strength of signals, the amount of markers bound on the probe surface can be determined.

In preferred forms of the invention, a single decision tree classification algorithm is utilized to analyze the data generated from SELDI. Algorithms used to generate such classifications are known in the art. For example, algorithms used to generate classification trees, such as from Classification Logic, based on cumulative probability, PeakMiner (Internet address: www.evms.edu/vpc/seld), or Classification And Regression Tree (CART) (Breiman, L., Friedman, J., Olshen, R., and Stone, C. J. (1984) Classification and Regression Trees Chapman and Hall, New York), and those developed by known methods that are suitable for the generation of such classification trees; for example, genetic cluster, logistical regression, surface vector machine, and neural nets can be used. (Jain et al. “Statistical Pattern Recognition: A Review,” IEEE Transactions on Pattern Analysis and Machine Intelligence, Vol. 22, No. 1, January 2000). For example, one such algorithm is more specifically described in Example 1 herein.

The test samples may be pre-treated prior to being subject to gas phase ion spectrometry. For example, the samples can be purified or otherwise pre-fractionated to provide a less complex sample for analysis. The optional purification procedure for the biomolecules present in the test sample may be based on the properties of the biomolecules, such as size, charge and function. Methods of purification include centrifugation, electrophoresis, chromatography, dialysis or a combination thereof. As known in the art, electrophoresis may be utilized to separate the biomolecules in the sample based on size and charge. Electrophoretic procedures are well-known to the skilled artisan, and include isoelectric focusing, sodium dodecyl sulfate polyacrylamide gel electrophoresis (SDS-PAGE), agarose gel electrophoresis, and other known methods of electrophoresis.

The purification step may be accomplished by a chromatographic fractionation technique, including size fractionation, fractionation by charge and fractionation by other properties of the biomolecules being separated. As known in the art, chromatographic systems include a stationary phase and a mobile phase, and the separation is based upon the interaction of the biomolecules to be separated with the different phases. In preferred forms of the invention, column chromatographic procedures may be utilized. Such procedures include partition chromatography, adsorption chromatography, size-exclusion chromatography, ion-exchange chromatography and affinity chromatography. Such methods are well-known to the skilled artisan. In size-exclusion chromatography, it is preferred that the size fractionation columns exclude molecules whose molecular mass is greater than about 10,000 Da.

In a preferred form of the invention, the sample is purified or otherwise fractionated on a bio-chromatographic chip by retentate chromatography before gas phase ion spectrometry. A preferred chip is the Protein Chip™ available from Ciphergen Biosystems, Inc. (Palo Alto, Calif.). As described above, the chip or probe is adapted for use in a mass spectrometer. The chip comprises an adsorbent attached to its surface. This adsorbent can function, in certain applications, as an in situ chromatography resin. In operation, the sample is applied to the adsorbent in an eluant solution. Molecules for which the adsorbent has affinity under the wash condition bind to the adsorbent. Molecules that do not bind to the adsorbent are removed with the wash. The adsorbent can be further washed under various levels of stringency so that analytes are retained or eluted to an appropriate level for analysis. An energy absorbing molecule can then be added to the adsorbent spot to further facilitate desorption and ionization. The analyte is detected by desorption from the adsorbent, ionization and direct detection by a detector. Thus, retentate chromatography differs from traditional chromatography in that the analyte retained by the affinity material is detected, whereas in traditional chromatography, material that is eluted from the affinity material is detected.

The biomarkers of the present invention may also be detected, qualitatively or quantitatively, by an immunoassay procedure. The immunoassay typically includes contacting a test sample with an antibody that specifically binds to or otherwise recognizes a biomarker, and detecting the presence of a complex of the antibody bound to the biomarker in the sample. The immunoassay procedure may be selected from a wide variety of immunoassay procedures known to the art involving recognition of antibody/antigen complexes, including enzyme immunoassays, competitive or non-competitive, and including enzyme-linked immunosorbent assays (ELISA), radioimmunoassays (RIA), and Western blots, and use of multiplex assays, including use of antibody arrays, wherein several desired antibodies are placed on a support, such as a glass bead or plate, and reacted or otherwise contacted with the test sample. Such assays are well-known to the skilled artisan and are described, for example, more thoroughly in Antibodies: A Laboratory Manual (1988) by Harlow & Lane; Immunoassays: A Practical Approach, Oxford University Press, Gosling, J. P. (ed.) (2001) and/or Current Protocols in Molecular Biology (Ausubel et al.) which is regularly and periodically updated.

The antibodies to be used in the immunoassays described herein may be polyclonal antibodies and may be obtained by procedures which are well-known to the skilled artisan, including injecting purified biomarkers into various animals and isolating the antibodies produced in the blood serum. The antibodies may alternatively be monoclonal antibodies whose method of production is well-known to the art, including injecting purified biomarkers into a mouse, for example, isolating the spleen cells producing the anti-serum, fusing the cells with tumor cells to form hybridomas and screening the hybridomas. The biormarkers may first be purified by techniques similarly well-known to the skilled artisan, including the chromatographic, electrophoretic and centrifugation techniques described previously herein. Such procedures may take advantage of the protein biomarker's size, charge, solubility, affinity for binding to selected components, combinations thereof, or other characteristics or properties of the protein. Such methods are known to the art and can be found, for example, in Current Protocols in Protein Science, J. Wiley and Sons, New York, N.Y., Coligan et al. (Eds.) (2002); Harris, E. L. V., and S. Angal in Protein purification applications: a practical approach, Oxford University Press, New York, N.Y. (1990). Once the antibody is provided, a biomarker can be detected and/or quantitated by the immunoassays previously described herein.

Although specific procedures for immunoassays are well-known to the skilled artisan, generally, an immunoassay may be performed by initially obtaining a sample as previously described herein from a test subject. The antibody may be fixed to a solid support prior to contacting the antibody with a test sample to facilitate washing and subsequent isolation of the antibody/protein biomarker complex. Examples of solid supports are well-known to the skilled artisan and include, for example, glass or plastic in the form of, for example, a microtiter plate. Antibodies can also be attached to the probe substrate, such as the Proteinchip® arrays described herein.

After incubating the test sample with the antibody, the mixture is washed and the antibody-marker complex may be detected. The detection can be accomplished by incubating the washed mixture with a detection reagent, and observing, for example, development of a color or other indicator. Any detectable label may be used. The detection reagent may be, for example, a second antibody which is labeled with a detectable label. Exemplary detectable labels include magnetic beads (e.g., DYNABEADS™), fluorescent dyes, radiolabels, enzymes (e.g., horseradish peroxide, alkaline phosphatase and others commonly used in enzyme immunoassay procedures), and colorimetric labels such as colloidal gold, colored glass or plastic beads. Alternatively, the marker in the sample can be detected using an indirect assay, wherein, for example, a labeled antibody is used to detect the bound marker-specific antibody complex and/or in a competition or inhibition assay wherein, for example, a monoclonal antibody which binds to a distinct epitope of the biomarker is incubated simultaneously with the mixture. The amount of an antibody-marker complex can be determined by comparing to a standard.

Throughout the assays, incubation and/or washing steps may be required after each combination of reagents. Incubation steps can vary from about 5 seconds to several hours, preferably from about 5 minutes to about 24 hours. However, the incubation time will depend upon the particular immunoassay, biomarker, and assay conditions. Usually the assays will be carried out at ambient temperature, although they can be conducted over a range of temperatures, such as about 0° C. to about 40° C.

Kits are provided that may, for example, be utilized to detect the biomarkers described herein. The kits can, for example, be used to detect any one or more of the biomarkers described herein, which may advantageously be utilized for diagnosing or aiding in the diagnosis of HNSCC or in a negative diagnosis.

In one embodiment, a kit may include a substrate that includes an adsorbent thereon, wherein the adsorbent is preferably suitable for binding one or more protein biomarkers described herein, and instructions to detect the biomarker by contacting a test sample as described herein with the adsorbent and detecting the biomarker retained by the adsorbent. In certain embodiments, the kits may include an eluant, or instructions for making an eluant, wherein the combination of the eluant and the adsorbent allows detection of the protein biomarkers by, for example, use of gas phase ion spectrometry. Such kits can be prepared from the materials described herein.

In yet another embodiment, the kit may include a first substrate that includes an adsorbent thereon (e.g., a particle functionalized with an adsorbent) and a second substrate onto which the first substrate can be positioned to form a probe which is removably insertable into a gas phase ion spectrometer. In other embodiments, the kit may include a single substrate which is in the form of a removably insertable probe with adsorbents on the substrate. In yet another embodiment, the kit may further include a pre-fractionation spin column (e.g, K-30 size exclusion column).

The kit may further include instructions for suitable operating parameters in the form of a label or a separate insert. For example, the kit may have standard instructions informing a consumer or other individual how to wash the probe after a particular form of sample is contacted with the probe. As a further example, the kit may include instructions for pre-fractionating a sample to reduce the complexity of proteins in the sample.

In a further embodiment, a kit may include an antibody that specifically binds to the marker and a detection reagent. Such kits can be prepared from the materials described herein. The kit may further include pre-fractionation spin columns as described above, as well as instructions for suitable operating parameters in the form of a label or a separate insert.

In yet another aspect of the invention, methods of using a plurality of classifiers to make a probable diagnosis of HNSCC are provided. In one form of the invention, a method includes a) obtaining mass spectra from a plurality of samples from normal subjects and subjects diagnosed with HNSCC; b) applying a decision tree analysis to at least a portion of the mass spectra to obtain a plurality of weighted base classifiers, wherein the classifiers include a peak intensity value and an associated threshold value; and c) making a probable diagnosis of HNSCC or a negative diagnosis based on a linear combination of the plurality of weighted base classifiers. In certain forms of the invention, the method includes using the peak intensity value and the associated threshold value in linear combination to make a probable diagnosis of HNSCC or a negative diagnosis. The preferred algorithm and data treatment is more fully described in Example 1.

The methods of the present invention have other applications as well. For example, the biomarkers can be used to screen for compounds that modulate the expression of the biomarkers in vitro or in vivo, which compounds in turn may be useful in treating or preventing HNSCC in patients. In another example, the biomarkers can be used to monitor the response to treatments for HNSCC. In yet another example, the biomarkers can be used in heredity studies to determine if the subject is at risk for developing HNSCC.

Thus, for example, the kits of this invention could include a solid substrate having an cation exchange function, such as a protein biochip (e.g., a Ciphergen WCX2 Proteinchip array, e.g., Proteinchip array) and a sodium acetate buffer for washing the substrate, as well as instructions providing a protocol to measure the biomarkers of this invention on the chip and to use these measurements to diagnose HNSCC.

Compounds suitable for therapeutic testing may be screened initially by identifying compounds which interact with one or more biomarkers listed in Table 1. By way of example, screening might include recombinantly expressing a biomarker listed in Table 1, purifying the biomarker, and affixing the biomarker to a substrate. Test compounds would then be contacted with the substrate, typically in aqueous conditions, and interactions between the test compound and the biomarker are measured, for example, by measuring elution rates as a function of salt concentration. Certain proteins may recognize and cleave one or more biomarkers of Table 1, in which case the proteins may be detected by monitoring the digestion of one or more biomarkers in a standard assay, e.g., by gel electrophoresis of the proteins.

In a related embodiment, the ability of a test compound to inhibit the activity of one or more of the biomarkers of Table 1 may be measured. One of skill in the art will recognize that the techniques used to measure the activity of a particular biomarker will vary depending on the function and properties of the biomarker. For example, an enzymatic activity of a biomarker may be assayed provided that an appropriate substrate is available and provided that the concentration of the substrate or the appearance of the reaction product is readily measurable. The ability of potentially therapeutic test compounds to inhibit or enhance the activity of a given biomarker may be determined by measuring the rates of catalysis in the presence or absence of the test compounds. The ability of a test compound to interfere with a non-enzymatic (e.g., structural) function or activity of one of the biomarkers of Table I may also be measured. For example, the self-assembly of a multi-protein complex which includes one of the biomarkers of Table 1 may be monitored by spectroscopy in the presence or absence of a test compound. Alternatively, if the biomarker is a non-enzymatic enhancer of transcription, test compounds which interfere with the ability of the biomarker to enhance transcription may be identified by measuring the levels of biomarker-dependent transcription in vivo or in vitro in the presence and absence of the test compound.

Test compounds capable of modulating the activity of any of the biomarkers of Table 1 may be administered to patients who are suffering from or are at risk of developing HNSCC or other cancer. For example, the administration of a test compound which increases the activity of a particular biomarker may decrease the risk of HNSCC in a patient if the activity of the particular biomarker in vivo prevents the accumulation of proteins for HNSCC. Conversely, the administration of a test compound which decreases the activity of a particular biomarker may decrease the risk of HNSCC in a patient if the increased activity of the biomarker is responsible, at least in part, for the onset of HNSCC.

At the clinical level, screening a test compound includes obtaining samples from test subjects before and after the subjects have been exposed to a test compound. The levels in the samples of one or more of the biomarkers listed in Table 1 may be measured and analyzed to determine whether the levels of the biomarkers change after exposure to a test compound. The samples may be analyzed by mass spectrometry, as described herein, or the samples may be analyzed by any appropriate means known to one of skill in the art. For example, the levels of one or more of the biomarkers listed in Table I may be measured directly by Western blot using radio- or fluorescently-labeled antibodies which specifically bind to the biomarkers. Alternatively, changes in the levels of mRNA encoding the one or more biomarkers may be measured and correlated with the administration of a given test compound to a subject. In a further embodiment, the changes in the level of expression of one or more of the biomarkers may be measured using in vitro methods and materials. For example, human tissue cultured cells which express, or are capable of expressing, one or more of the biomarkers of Table 1 may be contacted with test compounds. Subjects who have been treated with test compounds will be routinely examined for any physiological effects which may result from the treatment. In particular, the test compounds will be evaluated for their ability to decrease disease likelihood in a subject. Alternatively, if the test compounds are administered to subjects who have previously been diagnosed with HNSCC, test compounds will be screened for their ability to slow or stop the progression of the disease.

### Computer Implementation

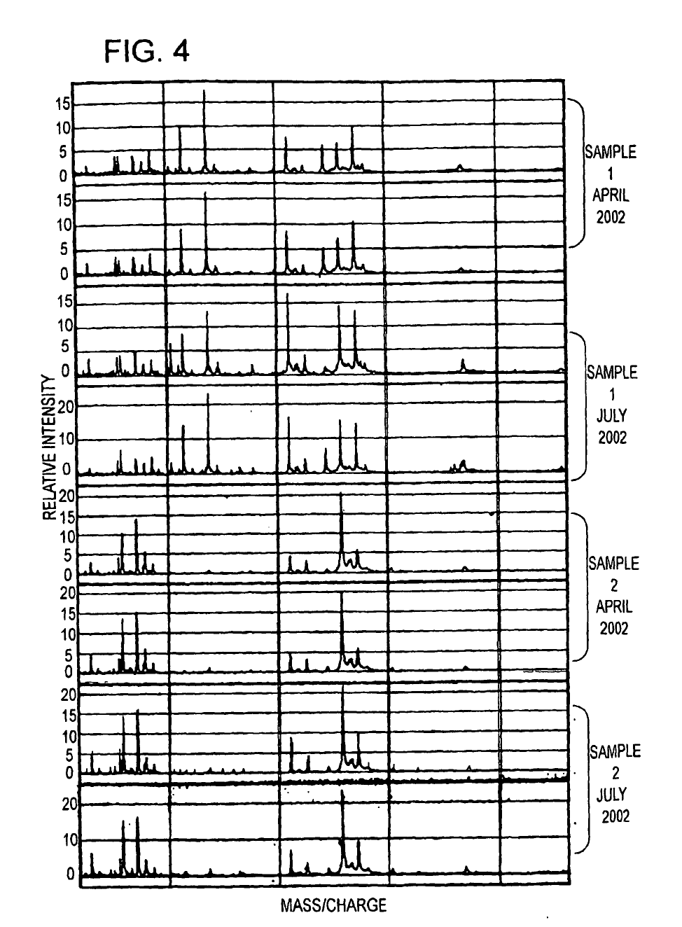

The techniques of the present invention may be implemented on a computing system 104 such as that depicted in FIG. 5. In this regard, FIG. 5 is an illustration of a computer system 104 which is also capable of implementing some or all of the computer processing in accordance with at least one computer implemented embodiment of the present invention.

Viewed externally, in FIG. 5, a computer system designated by reference numeral 104 has a computer portion 112 having drives 502 and 504, which are merely symbolic of a number of disk drives which might be accommodated by the computer system. Typically, these could include a floppy disk drive 502, a hard disk drive (not shown externally) and a CD ROM 504. The number and type of drives vary, typically with different computer configurations. Disk drives 502 and 504 are in fact optional, and for space considerations, can be omitted from the computer system.

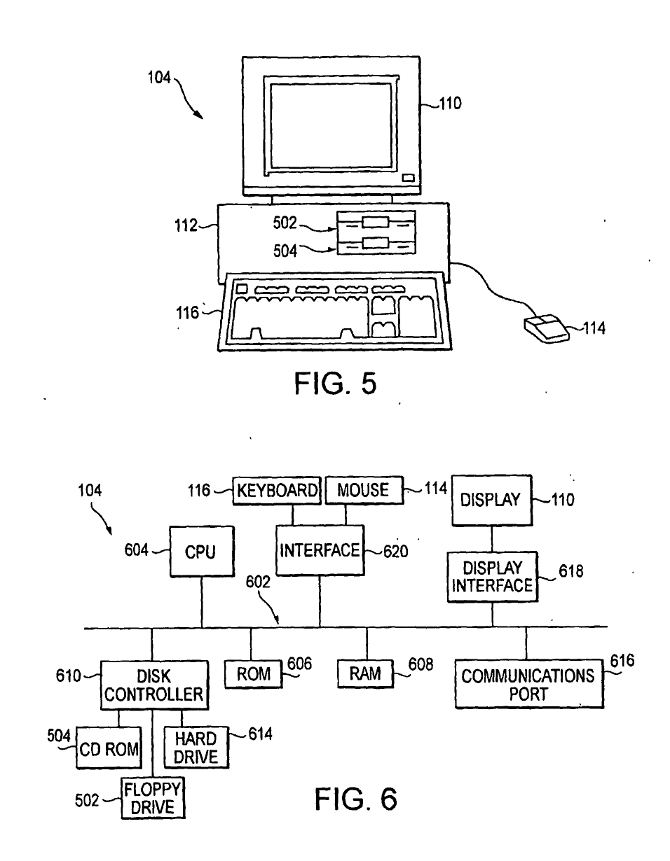

The computer system 104 also has an optional display monitor 110 upon which visual information pertaining to cells being normal or abnormal, suspected normal suspected abnormal, etc. can be displayed. In some situations, a keyboard 116 and a mouse 114 are provided as input devices through which input may be provided, thus allowing input to interface with the central processing unit (CPU) 604 (FIG. 6). Then again, for enhanced portability, the keyboard 116 can be either a limited function keyboard or omitted in its entirety. In addition, the mouse 114 optionally is a touch pad control device, or a track ball device, or even omitted in its entirety as well, and similarly may be used as an input device. In addition, the computer system 104 may also optionally include at least one infrared (or radio) transmitter and/or infrared (or radio) receiver for either transmitting and/or receiving infrared signals.

Although computer system 104 is illustrated having a single processor, a single hard disk drive 614 and a single local memory, the system 104 is optionally suitably equipped with any multitude or combination of processors or storage devices. Computer system 104 is, in point of fact, able to be replaced by, or combined with, any suitable processing system operative in accordance with the principles of the present invention, including hand-held, laptop/notebook, mini, mainframe and super computers, as well as processing system network combinations of the same.

FIG. 6 illustrates a block diagram of the internal hardware of the computer system 104 of FIG. 5. A bus 602 serves as the main information highway interconnecting the other components of the computer system 104. CPU 604 is the central processing unit of the system, performing calculations and logic operations required to execute a program. Read only memory (ROM) 606 and random access memory (RAM) 608 constitute the main memory of the computer system 104. Disk controller 610 interfaces one or more disk drives to the system bus 602. These disk drives are, for example, floppy disk drives such as 502, CD ROM or DVD (digital video disks) drive 504, or internal or external hard drives 614. As indicated previously, these various disk drives and disk controllers are optional devices.

A display interface 618 interfaces display 110 and permits information from the bus 602 to be displayed on the display 110. Again as indicated, display 110 is also an optional accessory. For example, display 110 could be substituted or omitted. Communications with external devices, for example, the other components of the system described herein, occur utilizing communication port 616. For example, optical fibers and/or electrical cables and/or conductors and/or optical communication (e.g., infrared, and the like) and/or wireless communication (e.g., radio frequency (RF), and the like) can be used as the transport medium between the external devices and communication port 616. Peripheral interface 620 interfaces the keyboard 116 and the mouse 114, permitting input data to be transmitted to the bus 602.

In alternate embodiments, the above-identified CPU 604, may be replaced by or combined with any other suitable processing circuits, including programmable logic devices, such as PALs (programmable array logic) and PLAs (programmable logic arrays). DSPs (digital signal processors), FPGAs (field programmable gate arrays), ASICs (application specific integrated circuits), VLSIs (very large scale integrated circuits) and the like.

Any presently available or future developed computer software language and/or hardware components can be employed in such embodiments of the present invention. For example, at least some of the functionality mentioned above could be implemented using Extensible Markup Language (XML), HTML, Visual Basic, C, C++, or any assembly language appropriate in view of the processor(s) being used. It could also be written in an interpretive environment such as Java and transported to multiple destinations to various users.

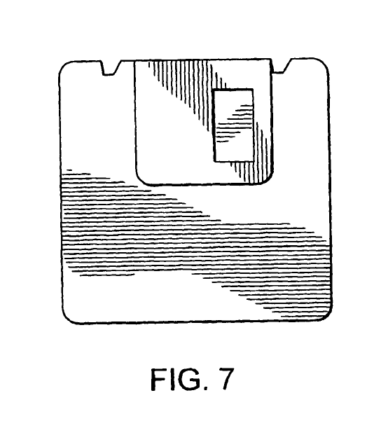

One of the implementations of the invention is as sets of instructions resident in the random access memory 608 of one or more computer systems 104 configured generally as described above. Until required by the computer system 104, the set of instructions may be stored in another computer readable memory, for example, in the hard disk drive 614, or in a removable memory such as an optical disk for eventual use in the CD-ROM 504 or in a floppy disk (e.g., floppy disk 702 of FIG. 7) for eventual use in a floppy disk drive 502. Further, the set of instructions (such as those written in Java, HTML, XML, Standard Generalized Markup Language (SGML), and/or Structured Query Language (SQL)) can be stored in the memory of another computer and transmitted via a transmission medium such as a local area network or a wide area network such as the Internet when desired by the user. One skilled in the art knows that storage or transmission of the computer program medium changes the medium electrically, magnetically, or chemically so that the medium carries computer readable information.

Reference will now be made to specific examples illustrating the biomarkers, kits, computer program media and methods above. It is to be understood that the examples are provided to illustrate preferred embodiments and that no limitation of the scope of the invention is intended thereby.

### EXAMPLE 1

Serum Samples

Serum samples were obtained from the Saint Louis University School of Medicine and the Pennsylvania State University College of Medicine. The serum procurement, data management, and blood collection protocols were approved by the Eastern Virginia Medical School Institutional Review Board. After informed consent, whole blood was drawn from head and neck cancer patients and from non-smoking controls. The serum was separated out, aliquotted, and frozen at −80° C. until thawed specifically for SELDI analysis.

Patient and Donor Cohorts

Specimens from two groups of patients were used in this study: 99 samples from patients diagnosed with HNSCC and 102 samples from normal, non-smoking control patients.

SELDI Protein Profiling

Serum samples were processed for SELDI analysis as previously described using the IMAC3 Proteinchip® pre-treated with CuSO₄ (Merchant, M., et al., Electrophoresis 21:1164-1177 (2000)). Briefly, 20 μl of serum was pre-treated with 8M urea, 1% CHAPS and vortexed for 10 minutes at 4° C. A further dilution was made in 1M urea, 0.125% CHAPS and PBS. Diluted serum was then added to the Proteinchips® with the aid of a bio-processor. Each serum sample was assayed in duplicate, with duplicate samples randomly placed on different Proteinchips®. Proteinchips® were then incubated at room temperature followed by washes of PBS and water. Arrays were allowed to air dry and a saturated solution of sinapinic acid (Ciphergen Biosystems, Fremont, Calif.) in 50% (v/v) acetonitrile, 0.5% (v/v) trifluoroacetic acid was added to each spot. The protein chip arrays were analyzed using the SELDI Proteinchip® System (PBS-II, Ciphergen Biosystems, Fremont, Calif.). Spectra were collected by the accumulation of 192 shots at laser intensity 220 in a positive mode. The protein masses were calibrated externally using purified peptide standards (Ciphergen Biosystems, Fremont, Calif.)

Data Analysis

Before analysis, the data was divided into two sets as follows: a training set consisting of 75 samples from each group (normal and HNSCC), and a test set of 24 HNSCC samples and 27 normal samples.

Peak Detection

Peak detection was performed using Ciphergen SELDI software version 3.0 (Internet address: www.ciphergen.com). The mass range from 2,000 Da to 21,000 Da was selected for analysis because this range contained the majority of the resolved proteins/peptides. Peak detection and clustering involved BioMarker Wizard Settings of signal to noise ratio of 3, a peak threshold of 10% of the spectra, and a cluster mass window of 0.2%.

All the labeled peaks (an average of 90 peaks/spectrum) were exported from SELDI to an Excel spreadsheet.

Classification and Regression Tree (CART) Analysis

Construction of the decision tree classification algorithm was performed as described by Breiman, L., et al., Classification and Regression Trees, (1984), using a training data set consisting of 150 samples (75 normal and 75 HNSCC). Details regarding the Classification and Regression Tree (CART) and the artificial intelligence bioinformatics algorithm incorporated within the BioMarker Patterns software program have also been described in Bertone, P., et al., Nucleic Acids Res. 29: 2884-2898 (2001); Kosuda, S., et al., Ann. Nucl. Med. 16:263-271 (2002). Classification trees split the data into two bins or nodes, using one rule at a time in the form of a question. The splitting decision was based on the presence or absence and the intensity levels of one peak. Therefore, each peak or cluster identified from the SELDI profile was a variable in the classification process. For example, the answer to “does mass A have an intensity less than or equal to X” splits the data into two nodes, a left node for yes and a right node for no. This “splitting” process continues until terminal nodes are reached and further splitting has no gain in data classification. Classification of terminal nodes was determined by the group (“class”) of samples (i.e., HNSCC, Normal) representing the majority of samples in that node. Classification trees were constructed using the training set, and following V-fold cross validation, the accuracy of each classification tree was challenged with the test set (blinded to the algorithm). Multiple classification trees were generated using this process, and the best performing tree was chosen for further testing.

Statistical Analysis

Specificity was calculated as the ratio of the number of negative samples correctly classified to the total number of true negative samples. Sensitivity was calculated as the ratio of the number of correctly classified diseased samples to the total number of diseased samples. Comparison of relative peak intensity levels between groups was calculated using the Student's t-test.

Data Analysis

Peak detection using the SELDI software program identified an average of 90 peaks/spectrum. Of these, 80 common peaks or clusters were generated from the training set, with masses ranging from 2,000 to 21,000 Daltons. Each cluster was determined with a mass window of 0.2% and represents one protein peak. As shown in Table 1 below, 32 of these peaks were found to have significant differential expression levels between HNSCC and control sera.

TABLE 1
Protein peaks differentially expressed in HNSCC vs. control serum

| m/z^{a} | P^{b} |
|---|---|
| 2778 | <0.0001 |
| 2951 | <0.0001 |
| 3772 | <0.05 |
| 3888 | <0.001 |
| 4181 | <0.02 |
| 4464 | <0.0001 |
| 5064 | <0.0001 |
| 5078 | <0.0001 |
| 5242 | <0.0001 |
| 5335 | <0.0001 |
| 5363 | <0.001 |
| 5544 | <0.01 |
| 5905 | <0.0001 |
| 5920 | <0.0001 |
| 6110 | <0.0001 |
| 7764 | <0.0001 |
| 7805 | <0.0001 |
| 7830 | <0.0001 |
| 7920 | <0.0001 |
| 7971 | <0.0001 |
| 8928 | <0.0001 |
| 9094 | <0.001 |
| 9134 | <0.0001 |
| 9181 | <0.0001 |
| 9287 | <0.001 |
| 9416 | <0.0001 |
| 10264 | <0.05 |
| 10843 | <0.05 |
| 11722 | <0.0001 |
| 11922 | <0.0001 |
| 13350 | <0.0001 |
| 14687 | <0.0001 |

^{a}m/z mass per charge
^{b}P value calculated from Student's t-test

Cart Analysis

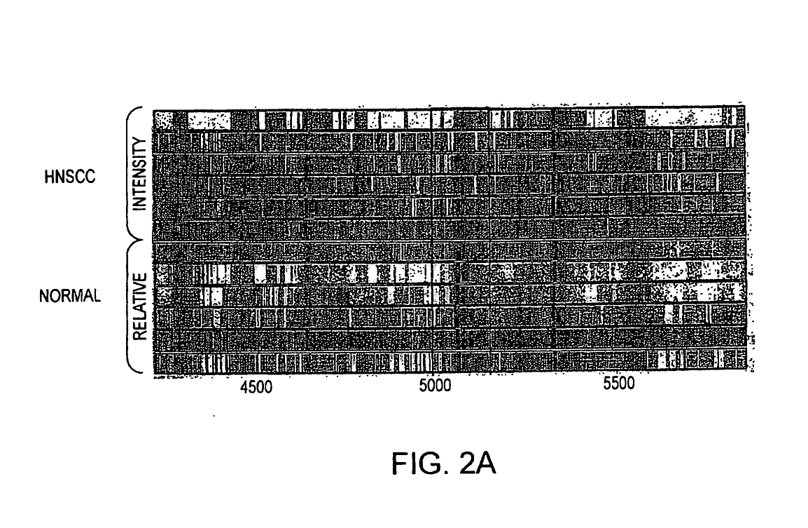

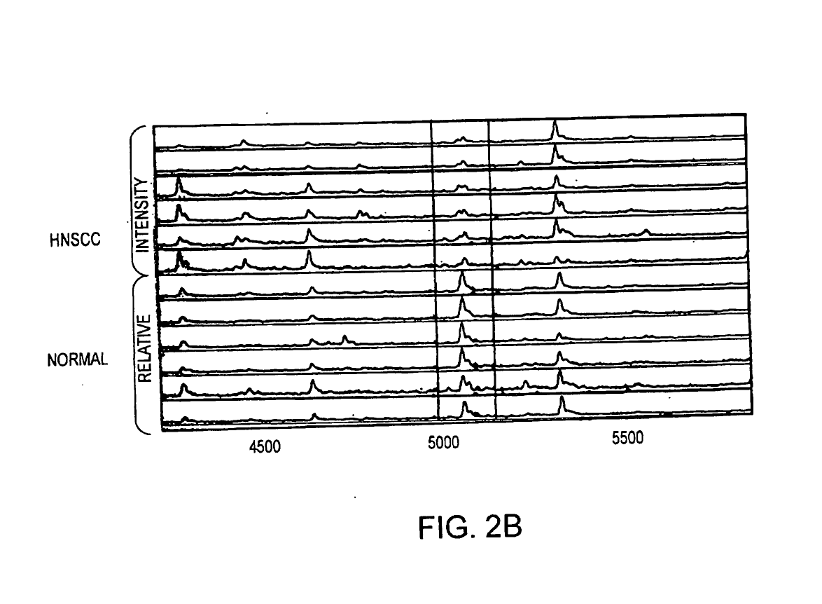

Using all 80 peaks, classification trees were created using the training set with V-fold cross validation. This type of cross validation uses random numbers to split up the data in the training set for testing each tree. Based on CART analysis, the underexpression of a protein peak at 5064 Daltons was used in all of the classification trees as the first primary splitter. FIG. 2 is a representative gel-view (2A) and spectra (2B) showing the underexpression of this peak in the HNSCC sera when compared to control sera. FIG. 3 shows the plotted averaged normalized intensity values for the 5064 Dalton peak and shows that the average expression is three-fold lower in HNSCC sera compared to the average expression in the control sera.

All 80 peaks were used to construct the decision tree classification algorithm. One sample classification algorithm used 3 masses between 5-16 kDa to generate 5 terminal nodes (FIG. 1). Once the algorithm identified the most discriminatory peaks, the classification rule was quite simple.

The most accurate tree correctly classified 90.7% of the HNSCC sera in the training set (see Table 2A) is classification tree algorithm was then challenged with a test set (blinded to the algorithm) consisting of 27 sera from healthy individuals and 24 sera from patients diagnosed with HNSCC (distinct from the training set). 100% of the controls and 83.3% of HNSCC samples were correctly identified (see Table 2B). The topology of the classification tree consisted of 3 primary peaks (5064, 13881, and 15139 Da) and 5 terminal nodes (see FIG. 1). A summation of the classification results from the 5 terminal nodes is presented for the training and test sets in Table 2 seen below.

TABLE 2
Decision Tree Classification of the HNSCC Training and Test Sets

| | | | |
|---|---|---|---|
| | | | Misclassified |
| Sample | Normal | HNSCC | Rate |

A. Training Set

| | | | | | | |
|---|---|---|---|---|---|---|
| Normal | 66 | 88.0% | 9 | 12.0% | 9 | 12.0% |
| (N = 75) | | | | | | |
| HNSCC | 7 | 9.3% | 68 | 90.7% | 7 | 9.3% |
| (N = 75) | | | | | | |
| Total | | | | | 16 | 10.7% |
| Samples | | | | | | |
| (N = 150) | | | | | | |

B. Test Set

| | | | | | | |
|---|---|---|---|---|---|---|
| Normal | 27 | 100.0% | 0 | 0.0% | 0 | 0.0% |
| (N = 27) | | | | | | |
| HNSCC | 4 | 16.7% | 20 | 83.3% | 4 | 16.7% |
| (N = 24) | | | | | | |
| Total | | | | | 4 | 7.8% |
| Samples | | | | | | |
| (N = 51) | | | | | | |

Reproducibility

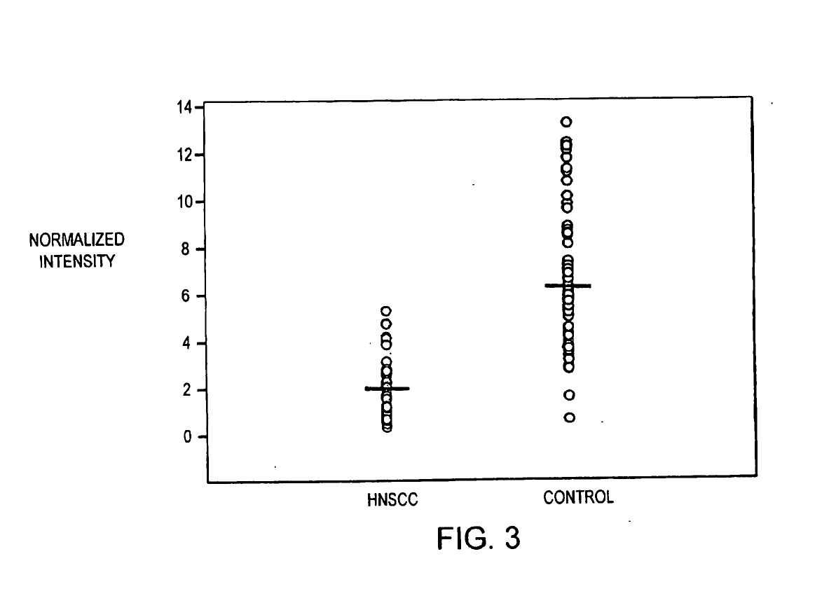

A key aspect of any clinical approach for reliable disease diagnostics and early detection is reproducibility. The reproducibility of SELDI data has been demonstrated previously using a pooled normal serum sample (Adam, B. L., et al., Cancer Res. 62:3609-3614 (2002)). The intra-assay and inter-assay coefficient of variance (CV) for peak masses is routinely 0.05% with normalized intensity CV values of 15-20%. To assess reproducibility, duplicate samples were assayed for each serum sample. FIG. 4 is an example of the reproducibility of the SELDI spectral data of sera run three months apart.

Discussion

Using SELDI/TOF-MS techniques, the present inventors have surprisingly achieved 100% specificity and 83.3% sensitivity for detection of HNSCC in a rapid and reproducible manner. While it has been observed that HNSCC is most often related to tobacco and alcohol use, control sera used in the preceding examples were obtained from normal individuals lacking those risk factors. In a preliminary study (data not shown), a classification tree such as described herein was tested with serum obtained from 100 healthy smokers (patients who have had a full head and neck examination without symptoms or findings) and achieved a sensitivity of 83% and specificity of 92.5% in differentiating patients with HNSCC from the healthy smokers. Significantly, the differences between healthy smokers and HNSCC patients were expected to be less than those between normal healthy controls and HNSCC patients, since progression from normal to cancer is multifocal and heterogeneous. This suggests that some “healthy” smokers may well be on the way to developing HNSCC without overt clinical signs.

Many protein peaks were found to be differentially expressed with high statistical significance in HNSCC compared to control sera (Table 1). It is notable that while not all of these significant peaks were used in the classification tree algorithms, the present invention contemplates the use of the differentially expressed markers. Unlike statistical tools that look only for single variables that can act as a predictor, CART analysis examines combinations of variables. A significant p-value may be achieved when testing for a group mean difference for a single protein peak. The classification algorithm is able to examine a number of different variables at once, looking for a combination of peak expression that gives the best classification. Furthermore, a peak without a significant p-value when tested between groups, may in fact be relevant to the classification algorithm. For instance, two of the peaks used in the best performing classification tree shown in FIG. 1 (13881 and 15139 Da) were individually not expressed differentially between the two groups of sera. However, they were significant to the classification tree to delineate subsets of groups that had been stratified by the significant peak at 5064 Da. The combination that resulted in maximum sensitivity/specificity for differentiating HNSCC from the non-cancer groups used the patterns of several different masses. One of these masses, the 5,064 Da peak, is under-expressed in HNSCC, yet was found in every classification tree generated with this set of sera, and is one example of how SELDI technology may aid both the discovery of new biologic markers for HNSCC as well as provide analysis of differences in protein expression patterns.

The use of the presently most preferred HNSCC classification system described herein relies on the protein “fingerprint” pattern of three masses: 5064±10.1; 13881±27.8; and 15139+30.3 Daltons. These masses have been found to be reproducibly and reliably detected. The mass values and the expression levels (i.e., the values of each peak) for these biomarkers enabled a correct classification or diagnosis. Importantly, knowing the identities of these biomarkers for the purpose of differential diagnosis is not required.

SELDI protein fingerprinting represents a paradigm shift from traditional cancer diagnostic approaches. The discovery of potentially new protein biomarkers is facilitated by SELDI/TOF-MS. While not intending to be bound by a particular theory, it appears that the protein pattern, rather than individual protein alteration, may be more important for differentiating normal healthy individuals from those who have, or are likely to develop, HNSCC. The high sensitivity and specificity achieved in this study using SELDI/TOF-MS techniques, coupled with a robust artificial intelligence classification algorithm, identified protein patterns in serum that distinguished healthy controls from HNSCC patients. This technique provides data that are easy to accumulate and should lend itself readily to clinical use.

While the invention has been illustrated and described in detail in the drawings and foregoing description, the same is to be considered as illustrative and not restrictive in character, it being understood that only the preferred embodiments have been shown and described and that all changes and modifications that come within the spirit of the invention are desired to be protected. In addition, all references and patents cited herein are indicative of the level of skill in the art and are hereby incorporated by reference in their entirety.

What is claimed is:

1. A method for aiding in a diagnosis of head and neck squamous cell carcinoma in a patient, comprising:
(a) obtaining a body fluid sample from a patient suspected of suffering from head and neck squamous cell carcinoma;
(b) detecting at least one protein biomarker in said sample, said protein biomarker selected from the group consisting of protein biomarkers having a molecular weight of about 2778±5.6, 2951±5.9, 3772±7.5, 3888±7.8, 4181±8.4, 4464±8.9, 5064±10.1, 5078±10.2, 5242±10.5, 5335±10.7, 5363±10.7, 5544±11.1, 5905±11.8, 5920±11.8, 6110±12.2, 7764±15.5, 7805±15.6, 7830±15.7, 7920±15.8, 7971±15.9, 8928±17.9, 9094±18.1, 9134±18.3, 9181±18.4, 9287±18.6, 9416±18.8, 10264±20.5, 10843±21.7, 11722±23.4, 11922±23.8, 13350±26.7, 13881±27.8, 14687±29.4, and 15139±30.3 Daltons;
(c) wherein said detecting of said at least one protein biomarker is correlated with a diagnosis of head and neck squamous cell carcinoma in said patient.

2. The method of claim 1, wherein said detection step further comprises identifying the differential expression of said at least one protein biomarker.

3. The method of claim 1, wherein the correlation takes into account the presence or absence of the said at least one protein biomarker in the sample and the frequency of detection of the same said at least one protein biomarker in a control.

4. The method of claim 3, wherein the correlation further takes into account the quantity of said at least one protein biomarker in the sample compared to a control quantity of the said at least one protein biomarker.

5. The method of claim 1, wherein at least one protein biomarker is selected from the group consisting of the about 5064±10.1, 13881±27.8, and 15139±30.3 Dalton biomarkers.

6. The method of claim 5, wherein said method comprises determining the quantity of the about 5064±10.1, 13881±27.8, and 15139±30.3 Dalton biomarkers.

7. The method of claim 1, wherein at least one protein biomarker is selected from the group consisting of the about 5064±10.1, 2778±5.6, 4464±8.9, and 3772±7.5 Dalton biomarkers.

8. The method of claim 7, wherein said method comprises determining the quantity of the about 5064±10.1, 2778±5.6, 4464±8.9, and 3772±7.5 Dalton biomarkers.

9. The method of claim 1, wherein at least one protein biomarker is selected from the group consisting of the about 5064±10.1, 5242±10.5, 13881±27.8, and 15139±30.3 Dalton biomarkers.

10. The method of claim 9, wherein said method comprises determining the quantity of the about 5064±10.1, 5242±10.5, 13881±27.8, and 15139±30.3 Dalton biomarkers.

11. The method of claim 1, wherein said detecting at least one protein biomarker is performed by mass spectrometry.

12. The method of claim 11, wherein said mass spectroscopy is laser desorption mass spectroscopy.

13. The method of claim of claim 12, wherein said mass spectroscopy is surface enhanced laser desorption/ionization mass spectroscopy.

14. The method of claim 13, wherein the laser desorption/ionization mass spectroscopy includes:
(a) providing a substrate comprising an adsorbent attached thereto;
(b) contacting the test sample with the adsorbent;
(c) desorbing and ionizing the biomarkers from the substrate; and
(d) detecting the desorbed/ionized biomarkers with a mass spectrometer.

15. The method of claim 14, further comprising purifying the test sample prior to contacting the sample with the adsorbent.

16. The method of claim 1, wherein said detecting at least one protein biomarker in a test sample from a subject is performed by immunoassay.

17. The method of claim 16, wherein said immunoassay is an enzyme immunoassay.

18. The method of claim 1, wherein the body fluid is blood serum.

19. The method of claim 1, wherein the body fluid is selected from the group consisting of seminal fluid, seminal plasma, saliva, blood, lymph fluid, lung/bronchial washes, mucus, feces, nipple secretions, sputum, tears, or urine.

20. The method of claim 1, wherein two to twenty biomarkers are detected.

21. The method of claim 1, wherein said method comprises:
(a) detecting the presence or absence of protein biomarkers having a molecular weight selected from the group consisting of about 5064±10.1, about 13881±27.8, and about 15139±30.3 Daltons; and
(b) correlating the detection with a probable diagnosis of head and neck squamous cell carcinoma.

22. The method of claim 21, wherein the presence or absence of the protein biomarker having a molecular weight of about 5064±10.1 Daltons is detected.

23. The method of claim 21, wherein the presence or absence of the protein biomarker having a molecular weight of about 5064±10.1 and about 13881±27.8 Daltons is detected.

24. The method of claim 21, wherein the presence or absence of the protein biomarker having a molecular weight of about 5064±10.1, about 13881±27.8, and about 15139±30.3 Daltons is detected.

25. The method of claim 21, wherein said detecting is performed by mass spectroscopy.

26. The method of claim 25, wherein said mass spectroscopy is laser desorption mass spectroscopy.

27. The method of claim 26, wherein said mass spectroscopy is surface enhanced laser desorption/ionization mass spectroscopy.

28. The method of claim 27, wherein the laser desorption/ionization mass spectroscopy includes:
(a) providing a substrate comprising an adsorbent attached thereto;
(b) contacting the test sample with the adsorbent;
(c) desorbing and ionizing the biomarkers from the substrate; and
(d) detecting the desorbed/ionized biomarkers with a mass spectrometer.

29. The method of claim 28, further comprising purifying the test sample prior to contacting the test sample with the adsorbent.

30. The method of claim 21, wherein said detecting is performed by an immunoassay.

31. The method of claim 30, wherein said immunoassay is an enzyme immunoassay.

32. A kit comprising a substrate comprising an adsorbent attached thereto, wherein the adsorbent is capable of retaining at least one protein biomarker selected from the group consisting of about 5064±10.1, 13881±27.8, and 15139±30.3 Daltons.

33. The kit of claim 32, wherein the substrate is a probe adapted for use with a gas phase ion spectrometer, said probe having a surface onto which the adsorbent is attached.

34. The kit of claim 32, wherein the adsorbent is a metal chelate adsorbent.

35. The kit of claim 32, wherein the adsorbent comprises a cationic group.

36. The kit of claim 32, wherein the substrate comprises a plurality of different types of adsorbent.

37. The kit of claim 32, wherein the adsorbent is an antibody that specifically binds to the biomarker.

38. The kit of claim 32, wherein the kit further comprises an eluant wherein the biomarker is retained on the adsorbent when washed with the eluant.

39. A kit comprising a substrate comprising an adsorbent attached thereto, wherein the adsorbent is capable of retaining at least one protein biomarker selected from the group consisting of about 2778±5.6, 2951±5.9, 3772±7.5, 3888±7.8, 4181±84, 4464±8.9, 5064±10.1, 5078±10.2, 5242±10.5, 5335±10.7, 5363±10.7, 5544±11.1, 5905±11.8, 5920±11.8, 6110±12.2, 7764±15.5, 7805±15.6, 7830±15.7, 7920±15.8, 7971±15.9, 8928±17.9, 9094±18.1, 9134±18.3, 9181±18.4, 9287±18.6, 9416±18.8, 10264±20.5, 10843±21.7, 11722±23.4, 11922±23.8, 13350±26.7, 13881±27.8, 14687±29.4, and 15139±30.3 Daltons.

40. The kit of claim 39, wherein the substrate is a probe adapted for use with a gas phase ion spectrometer, said probe having a surface onto which the adsorbent is attached.

41. The kit of claim 39, wherein the adsorbent is a metal chelate adsorbent.

42. The kit of claim 39, wherein the adsorbent comprises a cationic group.

43. The kit of claim 39, wherein the substrate comprises a plurality of different types of adsorbent.

44. The kit of claim 39, wherein the adsorbent is an antibody that specifically binds to the biomarker.

45. The kit of claim 39, wherein the kit further comprises an eluant wherein the biomarker is retained on the adsorbent when washed with the eluant.

46. A method of using a plurality of classifiers to make a probable diagnosis of head and neck squamous cell carcinoma or a negative diagnosis, comprising the steps of:
a) obtaining mass spectra from a plurality of samples from normal subjects and subjects diagnosed with head and neck squamous cell carcinoma; and;
b) applying a decision tree analysis to at least a portion of the mass spectra to obtain a plurality of weighted base classifiers comprising a peak intensity value and an associated threshold value, said values used in linear combination to make a probable diagnosis of at least one of head and neck squamous cell carcinoma and a negative diagnosis.

47. A computer program medium storing computer instructions therein for instructing a computer to perform a computer-implemented process of aiding in a diagnosis of benign prostate hyperplasia or prostate cancer, comprising:
(a) first computer program code means for detecting at least one protein biomarkers in a test sample from a subject, said protein biomarkers having a molecular weight selected from the group consisting of about 2778±5.6, 2951±5.9, 3772±7.5, 3888±7.8, 4181±8.4, 4464±8.9, 5064±10.1, 5078±10.2, 5242±10.5, 5335±10.7, 5363±10.7, 5544±11.1, 5905±11.8, 5920±11.8, 6110±12.2, 7764±15.5, 7805±15.6, 7830±15.7, 7920±15.8, 7971±15.9, 8928±17.9, 9094±18.1, 9134±18.3, 9181±18.4, 9287±18.6, 9416±18.8, 10264±20.5, 10843±21.7, 11722±23.4, 11922±23.8, 13350±26.7, 13881±27.8, 14687±29.4, and 15139±30.3 Daltons; and
(b) second computer program code means for correlating the detection with a probable diagnosis of benign prostate hyperplasia, prostate cancer or a negative diagnosis.

48. The medium of claim 47, wherein the at least one protein biomarker is the about 5064±10.1 Dalton protein biomarkers.

49. The medium of claim 47, wherein the protein biomarkers are the about 5064±10.1 and 13881±27.8 Dalton biomarkers.

50. The medium of claim 47, wherein the protein biomarkers are the about 5064±10.1, 13881±27.8, and 15139±30.3 Dalton biomarkers.

51. The method of claim 1, wherein the protein biomarker is about 5064±10.1 Daltons.

52. The method of claim 51, wherein said method comprises determining the quantity of the 5064±10.1 Dalton biomarker.

53. A method for aiding in a diagnosis of head and neck squamous cell carcinoma in a patient comprising:
(a) obtaining a body fluid sample from a patient suspected of suffering from head and neck squamous cell carcinoma;
(b) detecting, by surface enhanced laser desorption/ionization time of flight mass spectrometry (SELDI-TOF-MS), at least one protein biomarker in said sample, said protein biomarker selected from the group consisting of protein biomarkers having a molecular weight of about 5064±10.1, 13881±27.8, and 15139±30.3 Daltons;
(c) wherein said detecting of said at least one protein biomarker is correlated with a diagnosis of head and neck squamous cell carcinoma in said patient.

54. A method for aiding in a diagnosis of head and neck squamous cell carcinoma in a patient comprising:
(a) obtaining a body fluid sample from a patient suspected of suffering from head and neck squamous cell carcinoma;
(b) detecting, by surface enhanced laser desorption/ionization time of flight mass spectrometry (SELDI-TOF-MS), at least one protein biomarker in said sample, said protein biomarker selected from the group consisting of protein biomarkers having a molecular weight of about 5064±10.1, 2778±5.6, 4464±8.9, and 3772±7.5 Daltons;
(c) wherein said detecting of said at least one protein biomarker is correlated with a diagnosis of head and neck squamous cell carcinoma in said patient.

55. A method for aiding in a diagnosis of head and neck squamous cell carcinoma in a patient comprising:
(a) obtaining a body fluid sample from a patient suspected of suffering from head and neck squamous cell carcinoma;
(b) detecting, by surface enhanced laser desorption/ionization time of flight mass spectrometry (SELDI-TOF-MS), at least one protein biomarker in said sample, said protein biomarker selected from the group consisting of protein biomarkers having a molecular weight of about 5064±10.1, 5242±10.5, 13881±27.8, and 15139±30.3 Daltons;
(c) wherein said detecting of said at least one protein biomarker is correlated with a diagnosis of head and neck squamous cell carcinoma in said patient.

56. A method for aiding in a diagnosis of head and neck squamous cell carcinoma in a patient comprising:
(a) obtaining a body fluid sample from a patient suspected of suffering from head and neck squamous cell carcinoma;
(b) detecting, by surface enhanced laser desorption/ionization time of flight mass spectrometry (SELDI-TOF-MS), at least one protein biomarker in said sample, said protein biomarker selected from the group consisting of protein biomarkers having a molecular weight of about 5064±10.1, 5242±10.5, 13881±27.8, and 15139±30.3 Daltons;
(c) wherein said detecting of said at least one protein biomarker is correlated with a diagnosis of head and neck squamous cell carcinoma in said patient.

57. A method for aiding in a diagnosis of head and neck squamous cell carcinoma in a patient comprising:
(a) obtaining a body fluid sample from a patient suspected of suffering from head and neck squamous cell carcinoma;
(b) detecting, by surface enhanced laser desorption/ionization time of flight mass spectrometry (SELDI-TOF-MS), a protein biomarker in said sample having a molecular weight of about 5064±10.1 Daltons;
(c) wherein said detecting of said protein biomarker is correlated with a diagnosis of head and neck squamous cell carcinoma in said patient.

58. A method for aiding in a diagnosis of head and neck squamous cell carcinoma in a patient comprising:
(a) obtaining a body fluid sample from a patient suspected of suffering from head and neck squamous cell carcinoma;
(b) detecting, by surface enhanced laser desorption/ionization time of flight mass spectrometry (SELDI-TOF-MS), the quantity of a protein biomarker in said sample having a molecular weight of about 5064±10.1 Daltons;
(c) wherein underexpression of said protein biomarker is correlated with a diagnosis of head and neck squamous cell carcinoma in said patient.
